(19) United States
(12) Patent Application Publication
(10) Pub. No.: US 2026/0365920 A1
(43) Pub. Date: Nov. 19, 2026

(54) COMPOSITIONS AND METHODS FOR STABILIZING OXIDIZABLE SUBSTRATES USING ANTIOXIDANT

(71) Applicant: ECOLAB USA Inc., St. Paul, MN (US)

(72) Inventors: Ashish DHAWAN, Aurora, IL (US); Julianne TRUFFA, New Lenox, IL (US); Carter Martin SILVERNAIL, Lakeville, MN (US)

(73) Assignee: ECOLAB USA Inc., St. Paul, MN (US)

(21) Appl. No.: 19/663,907

(22) Filed: Apr. 30, 2026

Related U.S. Application Data
(60) Provisional application No. 63/799,007, filed on May 2, 2025.

(51) Int. Cl. C09K 15/08; C07D 317/64; C10L 1/185; C10M 105/38; C10M 105/40; C10M 129/20; C10M 169/04; C10N 30/10

(52) U.S. Cl. CPC C09K 15/08; C07D 317/64; C10L 1/1855; C10M 105/38; C10M 105/40; C10M 129/20; C10M 169/04; C10M 2207/046; C10M 2207/2835; C10M 2207/2895; C10N 2030/10

(57) ABSTRACT

The present disclosure relates to compositions and methods for stabilizing fluids, such as industrial fluids. The methods include adding a composition to a fluid. The compositions include a sesamol oligomer. The methods and compositions may be used in oxidizable industrial fluids, such as a biodiesel, a vegetable oil, a seed oil, a pyrolysis oil, a hydrocarbon, crude oil, a metal-working fluid, a lubricant, or any combination thereof.

### TECHNICAL FIELD

The present disclosure generally relates to compositions and methods for stabilizing oxidizable substrates.

### BACKGROUND

Antioxidants are used in various types of compositions and industries. For example, antioxidants are used to inhibit chemical oxidation of compositions obtained in the oil and gas industries. Antioxidants also play important roles as food preservatives and are often added to various food compositions to preserve food properties. A significant number of industrial antioxidants are synthetically produced from fossil oils, including sterically hindered phenols and alkylated diphenylamines. However, due to increasing concerns regarding their environmental and health impacts, there is an emerging demand for bio-based and bio-derived alternatives for industrial uses.

Hydrocarbon-containing compositions can benefit from use of antioxidants. In fats and oils, antioxidants can inhibit oxidation reactions, which can otherwise undesirably affect the chemistries of such fats and oils. Natural fats and oils include fatty acid portions that may have regions of unsaturation that are highly susceptible to oxidation. In food products, the oxidation of edible fats and oils can lead to unpleasant odors and tastes, causing spoilage of the food. Oxidation can be caused by exposure to oxygen and sunlight that lead to oxidation of hydrocarbons.

Antioxidants are also added to fuels, such as gasoline and gasoline/ethanol blends, and to lubricants to prevent oxidation of hydrocarbons therein. Gasoline fuels can oxidize easily upon exposure to conditions such as heat, oxygen, and ultraviolet light. Oxidation products result in gum or sediment within the fuel and thereby leading to problems such as plugging and corrosion of internal combustion engines. Antioxidants can prevent the polymerization of compounds in gasoline otherwise leading to residues that can damage internal combustion engines.

As an alternative to fuels and lubricants obtained from fossil sources, there has been interest in using renewable raw materials to produce biofuels and lubricants. In particular, biodiesel fuel and biolubricants have gained in popularity due to their performance properties, which can be similar and, in some cases, superior to petroleum-based fuels and lubricants. Biodiesel and biolubricants are typically derived from raw materials that are vegetable oils (i.e., glycerides), for example, soy, palm, rapeseed, sunflower, and coconut oils. These oils are typically converted to fatty acid alkyl esters by a transesterification reaction. Most commonly the transesterification process reacts the oils with methanol under basic catalysis to generate fatty acid methyl esters (FAMEs). While the use of biodiesel and biolubricants offers great commercial potential, there are various technical challenges associate with their preparation and use.

One challenge for the preparation and use of biodiesel and biolubricants is preserving the oxidation stability of these compositions. While vegetable oil raw materials have some protection from oxidation by naturally occurring antioxidants like tocopherols, the manufacturing and refining processes for biodiesel and biolubricants can remove these natural antioxidants.

Without antioxidants, a reduction in the oxidation stability of the biodiesel and biolubricants are susceptible to oxidative degradation. Oxidative biodiesel composition may deteriorate the properties of the biodiesel and cause problems with its use. Fatty acid esters in degradation can be caused and accelerated by factors such as contaminants like trace metals, contact with metal surfaces, aeration, and physical factors such as heat and ultraviolet light. For example, oxidation of biodiesel components by interaction with air and metal surfaces results in the formation of hydroperoxide radicals. These radicals promote free-radical chain reactions that cause the formation of oxidized chemical compounds of low molecular weight, like acids, aldehydes, and ketones. Polymeric gums are also formed as a result of oxidation. Polymeric gums lead to many problems, such as inadequate fuel combustion, deposits on engine parts such as injectors and pistons, and plugging of injectors and fuel lines. Therefore, oxidation not only reduces the effectiveness of the biodiesel, but it also causes long term mechanical problems in the engine and associated parts.

Antioxidants are used in biodiesel and biolubricants typically to prevent the oxidation of components therein, such as FAMEs. However, the use of known antioxidants can be challenging. Some conventional antioxidants need to be used at elevated concentrations to be useful as antioxidants, but often these antioxidants are not very effective in controlling peroxides formation as a result of interaction of the biodiesel with air. The antioxidant should be compatible with bio-based fuels and lubricants that are derived from a wide variety of plant-based materials and they should not demonstrate any adverse interactions with components in the bio-based compositions. Also, the antioxidant should be compatible with biodiesel blends, such as biodiesel that includes blends of (plant-based) biodiesel with petroleum-derived diesel. Also, added antioxidants should not adversely impact the properties of these materials, such as ignition and combustion properties, and also flow properties of biodiesel, especially flow properties of biodiesel at low temperatures.

Added antioxidants should also cause minimal environmental impact, especially when used with biodiesel, such as to not promote the formation of unwanted airborne particulates. Some conventional antioxidants include sulfur chemistries, the use of which can be undesirable from an environmental standpoint.

### BRIEF SUMMARY

In some aspects, the present disclosure provides methods of stabilizing an oxidizable substrate, such as an oxidizable fluid. Stabilizing may include preventing or inhibiting oxidation of a substrate. In one example, the methods described herein may comprise adding an effective amount of a composition to the oxidizable substrate (e.g., oxidizable fluid), the composition comprising a sesamol oligomer.

In some aspects, the present disclosure provides a sesamol oligomer of formula I:

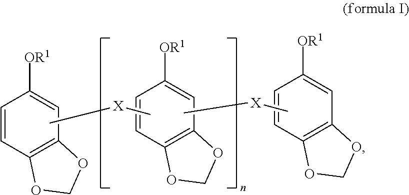

or a salt thereof, wherein each of R¹, X, and n is as described herein.

The present disclosure also provides an antioxidant, such as a bio-derived antioxidant, for industrial uses. In some aspects, sesamol is a precursor for providing an antioxidant, such as a sesamol oligomer as described herein.

The present disclosure also provides an effective antioxidant for a broad spectrum of oxidizable substrates, such as oxidizable fluids like a biodiesel, a vegetable oil, a seed oil, a pyrolysis oil, a hydrocarbon, crude oil, a metal-working fluid, a lubricant, or any combination thereof. Other oxidizable substrates include, but are not limited to, plastics, polymers, elastomer, tires and cables.

In some aspects, the present disclosure provides a composition including an oxidizable substrate, such as an oxidizable fluid, and an antioxidant, such as a sesamol oligomer. The oxidizable fluid, for example, may be a biodiesel, a vegetable oil, a seed oil, a pyrolysis oil, a hydrocarbon, crude oil, a metal-working fluid, a lubricant, or any combination thereof.

The foregoing has outlined rather broadly the features and technical advantages of the present disclosure in order that the detailed description that follows may be better understood. Additional features and advantages of the disclosure will be described hereinafter that form the subject of the claims of this application. It should be appreciated by those skilled in the art that the conception and the specific embodiments disclosed may be readily utilized as a basis for modifying or designing other embodiments for carrying out the same purposes of the present disclosure. It should also be realized by those skilled in the art that such equivalent embodiments do not depart from the spirit and scope of the disclosure as set forth in the appended claims.

### BRIEF DESCRIPTION OF THE SEVERAL VIEWS OF THE DRAWINGS

A detailed description of the invention is hereafter described with specific reference being made to the drawings in which:

FIG. 1 shows the oxidation induction time (minutes) versus concentration (ppm) curves of sesamol and sesamol oligomer for Radialube 7364 lubricant oil; and

FIG. 2 shows the oxidation induction time (minutes) versus concentration (ppm) curves for sesamol and sesamol oligomer in pyrolysis oil-1.

### DETAILED DESCRIPTION

Although the present disclosure provides references to illustrative embodiments, persons skilled in the art will recognize that changes may be made in form and detail without departing from the spirit and scope of the invention. Reference to various embodiments does not limit the scope of the claims attached hereto. Additionally, any examples set forth in this specification are not intended to be limiting and merely set forth some of the many possible embodiments for the appended claims.

Additional advantages and novel features of the present disclosure will be set forth in part in the description that follows, and in part will become apparent to those skilled in the art upon examination of the following, or may be learned through routine experimentation upon practice of the present disclosure.

Unless otherwise defined, all technical and scientific terms used herein have the same meaning as commonly understood by one of ordinary skill in the art. In case of conflict, the present document, including definitions, will control. Methods and materials are described below, although methods and materials similar or equivalent to those described herein can be used in practice or testing of the present application. All publications, patent applications, patents and other references mentioned herein are incorporated by reference in their entirety.

The present disclosure provides compositions and methods for stabilizing an oxidizable substrate, such as an oxidizable fluid. The stability of the oxidizable substrate can be improved by additives that inhibit, prevent or reduce gum formation, discoloration and oxidation. In some embodiments, stability is achieved by use of stabilizers, such as antioxidants. Compositions comprising antioxidants are disclosed herein and may be used in methods for stabilizing oxidizable substrates, such as oxidizable fluids, by preventing or inhibiting oxidation of the substrate.

In some embodiments, the present disclosure provides an antioxidant, such as a bio-derived antioxidant. In some embodiments, sesamol, a phenolic natural compound present in sesame seeds and oil, is a precursor for providing an antioxidant. Sesamol, for example, is known for its potent antioxidant properties in biological systems and plays a significant role in protecting cells from oxidative stress by neutralizing free radicals.

The term “antioxidant” as used herein is a compound that can inhibit, prevent or reduce oxidation, deterioration, degradation and gum formation. Antioxidants are capable of acting as scavengers preventing free radical formation. In one example, an antioxidant is a stabilizer.

The term “oligomer” refers to a compound including two or more (e.g., 2 to 40, such as 2 to 30, 2 to 20, or 2 to 10) monomer units. An oligomer with a specific number of monomer units is referred to by the Greek prefix denoting that number, with the ending-mer: thus dimer, trimer, tetramer, pentamer, and hexamer refer to molecules with two, three, four, five, and six units, respectively. An oligomer may include a dimer, a trimer, or a combination thereof.

The term “oxidative stability” as used herein refers to the susceptibility of a fluid to oxidative degradation. Oxidative stability may be measured by oxidation stability index (OSI), oxidation induction time (OIT), or oxidation temperature (OT).

The term “oxidizable substrate” refers to a composition comprising one or more components that are susceptible to oxidative degradation. Examples of oxidizable substrates include fluids, plastics, polymers, elastomer, tires, cables, and the like.

The term “sesamol” as used herein refers to a compound of the structure:

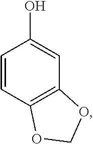

or a salt thereof. Sesamol is a phenolic natural compound present in sesame seeds and oil. It will be understood that sesamol preferably refers to a structure with a free hydroxy group (—OH) or salt thereof, however, in some instances, may also refer to a structure with a protected hydroxy group (—OH) or salt thereof.

The term “sesamol oligomer” refers to a compound including two or more monomer units of sesamol.

The term “stabilizer” as used herein refers to a composition that inhibits, prevents, and/or reduces oxidation, discoloration, deterioration, and/or degradation of the oxidizable fluid feedstock, formation or settling out of insoluble products (e.g., gums), or combinations thereof.

The presently disclosed term “stabilizer” may be used to cover both “stabilizer” compounds as well as “antioxidant” compounds. In some embodiments, however, the term may be used to cover only “stabilizer” compounds or “antioxidant” compounds, and such embodiments will be explicitly indicated as such.

The terms “C_{x-y}” or “Cₓ-C_{y}”, when used in conjunction with a chemical moiety, such as, alkyl, alkenyl, alkynyl, acyl, acyloxy, or alkoxy are meant to include groups that contain from x to y carbons in the chain. C₀ alkyl indicates a hydrogen where the group is in a terminal position or a bond if internal. A C₁₋₆ alkyl group, for example, contains from one to six carbon atoms in the chain.

The term “alkyl” refers to a straight- or branched-chain monovalent hydrocarbon group. The term “alkylene” refers to a straight- or branched-chain divalent hydrocarbon group. In some embodiments, it can be advantageous to limit the number of atoms in an “alkyl” or “alkylene” to a specific range of atoms, such as C₁-C₃₆ alkyl or C₁-C₃₆ alkylene, C₁-C₁₈ alkyl or C₁-C₁₈ alkylene, or C₁-C₆ alkyl or C₁-C₆ alkylene. Unless otherwise indicated, an alkyl or alkylene group as described herein alone or as part of another group is an optionally substituted linear or branched saturated hydrocarbon chain containing from, for example, one to about thirty-six carbon atoms or one to about eighteen carbon atoms, in the main chain. Examples of alkyl groups include methyl, ethyl, n-propyl, i-propyl, n-butyl, i-butyl, s-butyl, t-butyl, n-pentyl, i-pentyl, s-pentyl, t-pentyl, and the like. Examples of alkylene groups include methylene (—CH₂—), ethylene ((—CH₂—)₂), n-propylene ((—CH₂—)₃), iso-propylene ((—C(H)(CH₃)CH₂—)), n-butylene ((—CH₂—)₄), and the like. An alkyl or alkylene group can be substituted with any of the substituents in the various embodiments described herein, including one or more of such substituents, such as hydroxy groups.

The term “alkenyl” refers to a straight- or branched-chain mono-valent hydrocarbon group having one or more double bonds. In some embodiments, it can be advantageous to limit the number of atoms in an “alkenyl” to a specific range of atoms, such as C₂-C₆₀ alkenyl, C₂-C₃₆ alkenyl, C₂-C₁₈ alkenyl, or C₂-C₆ alkenyl. Unless otherwise indicated, an alkenyl group as described herein alone or as part of another group is an optionally substituted linear or branched hydrocarbon chain containing from, for example, one to about sixty carbon atoms, such as one to about thirty-six carbon atoms or one to about eighteen carbon atoms, in the main chain. Examples of alkenyl groups include ethenyl (or vinyl), allyl, and but-3-en-1-yl. Included within this term are cis and trans isomers and mixtures thereof. A double bond of an alkenyl group may be represented by the structure “ [symbol] ” or “ [symbol] .” It will be appreciated that a double bond represented by the structure “ [symbol] ” may represent an unspecified cis or trans configuration or a mixture of cis and trans configurations. It will be appreciated that an alkenyl can be unsubstituted or substituted as described herein. An alkenyl group can be substituted with any of the substituents in the various embodiments described herein, including one or more of such substituents, such as hydroxy groups.

The term “cycloalkyl” refers to a monovalent cyclic alkyl group containing from, for example, about 3 to about 12 carbon atoms, preferably from about 4 to about 8 carbon atoms, and more preferably from about 4 to about 6 carbon atoms. The term “cycloalkylene” refers to a divalent cyclic alkyl group containing from, for example, about 3 to about 12 carbon atoms, preferably from about 4 to about 8 carbon atoms, and more preferably from about 4 to about 6 carbon atoms. Examples of such cycloalkyl and cycloalkylene groups include cyclopropyl, cyclobutyl, cyclopentyl, cyclohexyl, cycloheptyl, cyclooctyl, and the like. Examples of such cycloalkylene groups include cyclopropylene, cyclobutylene, cyclopentylene, cyclohexylene, cycloheptylene, cyclooctylene, and the like. A cycloalkyl or cycloalkylene group can be substituted with any of the substituents in the various embodiments described herein, including one or more of such substituents. For example, the cycloalkyl or cycloalkylene groups may be unsubstituted or further substituted with hydroxy groups or alkyl groups, such as methyl groups, ethyl groups, and the like.

The term “aryl” includes monovalent substituted or unsubstituted aromatic groups in which each atom of the ring is carbon. The term “arylene” includes bivalent substituted or unsubstituted aromatic groups in which each atom of the ring is carbon. Preferably the ring is a 5- to 10-membered ring, more preferably a 6-membered (e.g., phenyl) or a 10-membered (e.g., naphthyl) ring. The terms “aryl” and “arylene” also include polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. The terms “aryl” and “arylene” apply to cyclic groups that are planar and comprise 4n+2 electrons, according to Huckel's Rule. The term “aryl” includes both unsubstituted and substituted aryl groups, the latter of which refers to aryl moieties having substituents replacing a hydrogen. Examples of such aryl groups include phenyl, naphthyl, benzofuran, indole, benzodioxole, chromone, and the like. Representative examples of substituted aryl groups include, but are not limited to, hydroxyphenyl, carboxyphenyl, methoxyphenyl, and 4-hydroxy-3-methoxyphenyl.

The term “alkoxy” refers to an alkyl group having an oxygen attached thereto. Representative alkoxy groups include methoxy, ethoxy, propoxy, tert-butoxy and the like.

The term “halo” or “halogen” refers to fluorine, chlorine, bromine, or iodine.

The terms “hydroxy” and “hydroxyl” refer to a group represented by —OH.

Compounds of the present disclosure may be substituted with suitable substituents. The term “suitable substituent,” as used herein, is intended to mean a chemically acceptable functional group, preferably a moiety that does not negate the activity of the compounds. Such suitable substituents include, but are not limited to, halo groups, alkyl groups, alkenyl groups, alkynyl groups, hydroxy groups, oxo groups, mercapto groups, alkylthio groups, alkoxy groups, aryl or heteroaryl groups, aryloxy or heteroaryloxy groups, aralkyl or heteroaralkyl groups, aralkoxy or heteroaralkoxy groups, carboxy groups, heterocylic groups, cycloalkyl groups, amino groups, alkyl- and dialkylamino groups, carbamoyl groups, alkylcarbonyl groups, alkoxycarbonyl groups, alkylaminocarbonyl groups, dialkylamino carbonyl groups, arylcarbonyl groups, aryloxy-carbonyl groups, alkylsulfonyl groups, and arylsulfonyl groups. In some embodiments, suitable substituents may include hydroxy, a C₁-C₆ alkyl group optionally substituted with a hydroxy, a C₃-C₂₀ cycloalkyl group optionally substituted with a hydroxy, or a C₆-C₁₀ aryl group optionally substituted with a hydroxy. Those skilled in the art will appreciate that many substituents can be substituted by additional substituents.

The term “substituted” as in “substituted alkyl,” means that in the group in question (i.e., the alkyl group), at least one hydrogen atom bound to a carbon atom is replaced with one or more substituent groups, such as hydroxy (—OH), alkylthio, phosphino, amido (—CON(R_{A})(R_{B}), wherein R_{A} and R_{B} are independently hydrogen, alkyl, or aryl), amino (—N(R_{A})(R_{B}), wherein R_{A} and R_{B} are independently hydrogen, alkyl, or aryl), halo (fluoro, chloro, bromo, or iodo), silyl, nitro (—NO₂), an ether (—OR_{A} wherein R_{A} is alkyl or aryl), an ester (—OC(O)R_{A} wherein R_{A} is alkyl or aryl), keto (—C(O)R_{A} wherein R_{A} is alkyl or aryl), heterocyclo, and the like.

When the term “substituted” introduces a list of possible substituted groups, it is intended that the term apply to every member of that group. That is, the phrase “optionally substituted alkyl or aryl” is to be interpreted as “optionally substituted alkyl or optionally substituted aryl.”

As used herein, the terms “salt” or “or salt thereof” are used to refer to an acid addition salt or a base addition salt of compounds of present disclosure. The selection of the appropriate salt will be known to a person skilled in the art.

It has been discovered that fluids, such as industrial fluids, can be stabilized using a composition comprising, consisting essentially of, or consisting of a sesamol oligomer. For example, the oxidizable fluids can be stabilized using an effective amount of a sesamol oligomer.

In some embodiments, the present disclosure provides methods of stabilizing an oxidizable substrate, such as an oxidizable fluid. In some embodiments, the method comprises adding a composition comprising a sesamol oligomer to the oxidizable fluid. In some embodiments, the method comprises producing a sesamol oligomer and adding a composition comprising the sesamol oligomer to the oxidizable fluid. In some embodiments, the step of producing the sesamol oligomer comprises reacting sesamol with an aldehyde in the presence of a catalyst.

In some embodiments, the sesamol oligomer comprises a sesamol dimer, a sesamol trimer, or a combination thereof.

In some embodiments, the sesamol oligomer is of formula I:

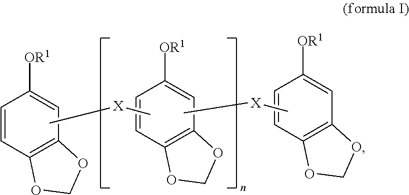

or a salt thereof, wherein: each R¹ is independently hydrogen or alkyl (e.g., C₁-C₆ alkyl), each X is independently a bond or alkylene (e.g., C₁-C₆ alkylene), and n is 0 or an integer of 1 to 20.

In some embodiments, the sesamol oligomer is of formula Ia:

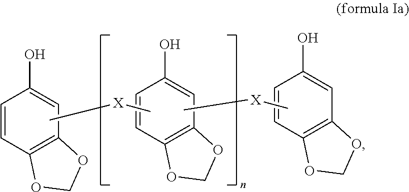

or a salt thereof, wherein each of X and n is as described herein.

In some embodiments, the sesamol oligomer of formula I comprises a sesamol dimer of formula II:

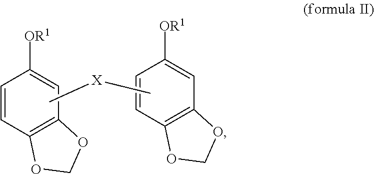

or a salt thereof, wherein each of R¹ and X is as described herein.

In some embodiments, the sesamol oligomer of formula I comprises a sesamol dimer of formula IIa:

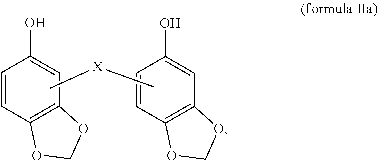

or a salt thereof, wherein X is as described herein.

In some embodiments, the sesamol oligomer of formula I comprises a sesamol trimer of formula III:

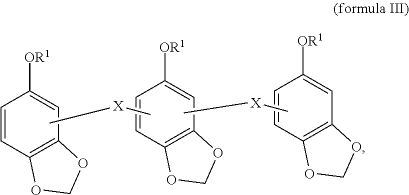

or a salt thereof, wherein each of R¹ and X is as described herein.

In some embodiments, the sesamol oligomer of formula I comprises a sesamol trimer of formula IIIa:

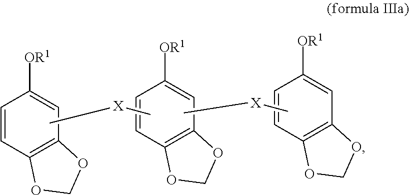

or a salt thereof, wherein each X is as described herein.

In some embodiments, the sesamol oligomer of formula I or Ia comprises a sesamol dimer of formula II or IIa, a sesamol trimer of formula III or IIa, or any combination thereof.

In some embodiments of formula I, Ia, II, IIa, III or IIIa, each R¹ is independently hydrogen or C₁-C₆ alkyl. In some embodiments, each R¹ is independently hydrogen, methyl, or ethyl. In some embodiments, each R¹ is hydrogen.

In some embodiments of formula I, Ia, II, IIa, III or IIIa, each X is independently a bond or C₁-C₆ alkylene (e.g., substituted or unsubstituted C₁-C₆ alkylene). In some embodiments, each X is independently a bond, methylene (e.g., substituted or unsubstituted methylene), or ethylene (e.g., substituted or unsubstituted ethylene). In some embodiments, each X is independently methylene (e.g., substituted or unsubstituted methylene) or ethylene (e.g., substituted or unsubstituted ethylene). In some embodiments, each X is methylene, for example, substituted or unsubstituted methylene.

In some embodiments, each X is C₁-C₆ alkylene (e.g., substituted or unsubstituted C₁-C₆ alkylene), wherein each hydrogen atom on C₁-C₆ alkylene is independently optionally replaced by hydroxy, halogen, C₁-C₆ alkoxy, or C₆-C₁₀ aryl. In some embodiments, each X is unsubstituted C₁-C₆ alkylene (e.g., substituted or unsubstituted C₁-C₆ alkylene), or C₁-C₆ alkylene substituted with hydroxy, halogen, C₁-C₆ alkoxy, or C₆-C₁₀ aryl.

In some embodiments of formula I, Ia, II, IIa, III or IIIa, X is a divalent linker. In some embodiments, each X is bound to a carbon atom ortho to OR¹ (e.g., OH). In some embodiments, X is bound to a first carbon atom ortho to OR¹ on a first sesamol monomer and a second carbon atom ortho to OR¹ on a second sesamol monomer. It will be understood that the phrase “each X is bound to a carbon atom ortho to OR¹ (e.g., OH)” means that each X is bound to sesamol at either of the positions of the representative structures:

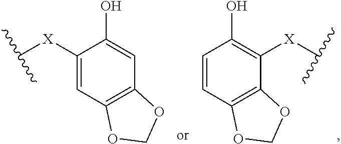

wherein “ [symbol] ” represents a point of attachment to another sesamol.

In some embodiments of formula I or Ia, n is 0 or an integer of 1 to 20. In some embodiments, n is 0 or an integer of 1 to 5. In some embodiments, n is 0 or 1.

In some embodiments, the sesamol oligomer of formula I is selected from the group consisting of:

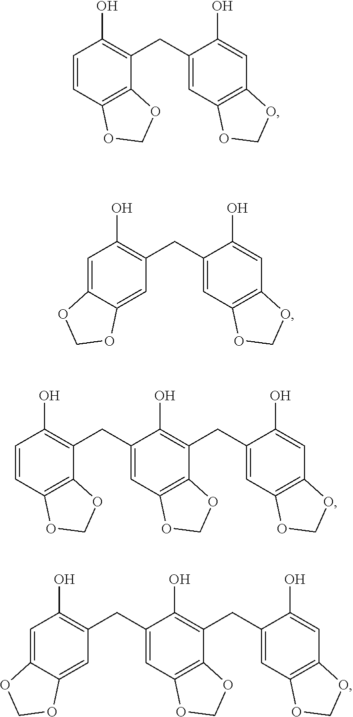 [SMILES: Oc1cc2c(cc1Cc1c(O)ccc3c1OCO3)OCO2 ; Oc1cc2c(cc1Cc1cc3c(cc1O)OCO3)OCO2 ; Oc1cc2c(cc1Cc1c(O)c(Cc3c(O)ccc4c3OCO4)cc3c1OCO3)OCO2 ; Oc1cc2c(cc1Cc1cc3c(c(Cc4cc5c(cc4O)OCO5)c1O)OCO3)OCO2]

and combinations thereof.

In some embodiments, the sesamol oligomer is provided by reacting sesamol and an aldehyde, optionally in the presence of a catalyst.

In some embodiments, the method includes producing the sesamol oligomer by contacting (e.g., reacting) sesamol with an aldehyde, optionally in the presence of a catalyst. In some embodiments, the method includes contacting sesamol with an aldehyde, optionally in the presence of a catalyst, thereby producing a sesamol oligomer.

In some embodiments, the step of contacting may be carried out using a molar ratio of sesamol to aldehyde of about 5:1 to about 1:5, for example, about 4:1 to about 1:4, about 3:1 to about 1:3, about 2:1 to about 1:2, about 4:1 to about 1:1, about 3:1 to about 1:1, about 2:1 to about 1:1, about 1:1 to about 1:4, about 1:1 to about 1:3, about 1:1 to about 1:2, or about 1:1.

In some embodiments, the step of contacting may be carried out at a temperature of about 75 °C to about 150 °C, for example, about 75 °C to about 125 °C, about 75 °C to about 100 °C, about 90 °C to about 150 °C, about 90 °C to about 125 °C, or about 90 °C to about 100 °C.

In some embodiments, the step of contacting may be carried out for about 2 hours to about 48 hours, for example, about 6 hours to about 24 hours, about 6 hours to about 18 hours, about 12 hours to about 24 hours, or about 12 hours to about 18 hours.

In some embodiments, the catalyst is an acid (i.e., an acid catalyst) or a base (i.e., a base catalyst). The catalyst is not particularly limited, and any suitable catalyst may be employed. In some embodiments, the catalyst is an acid catalyst selected from the group consisting of oxalic acid, hydrochloric acid, orthophosphoric acid, sulfuric acid, p-toluene-sulfonic acid, and any combination thereof. In some embodiments, the catalyst is a base catalyst selected from the group consisting of sodium hydroxide, potassium hydroxide, lithium hydroxide, and any combination thereof.

In some embodiments, the aldehyde is formaldehyde, acetaldehyde, benzaldehyde, vanillin, salicylaldehyde, glyoxal, glyoxylic acid, and any combination thereof. The aldehyde is not particularly limited, and any suitable aldehyde source may be employed. Formaldehyde, for example, may be provided as formalin, paraformaldehyde, or combinations thereof.

In some embodiments, the method includes heating the oxidizable fluid to a temperature of about 20 °C to about 250 °C. For example, the heating may be carried out at temperatures less than about 250 °C, less than about 200 °C, about 20 °C to about 200 °C, about 20 °C to about 175 °C, about 20 °C to about 150 °C, about 20 °C to about 125 °C, about 20 °C to about 100 °C, about 50 °C to about 250 °C, about 50 °C to about 200 °C, about 50 °C to about 175 °C, about 50 °C to about 150 °C, about 50 °C to about 125 °C, about 50 °C to about 100 °C, about 100 °C to about 250 °C, about 100 °C to about 200 °C, about 100 °C to about 175 °C, about 100 °C to about 150 °C, or about 100 °C to about 125 °C.

In some embodiments, the composition comprises an additional component selected from the group consisting of a solvent, an additional antioxidant, and combinations thereof.

If a composition of the present disclosure comprises an additional component, the amount of additional component in the composition is not particularly limited. For example, a composition of the present disclosure may comprise from about 0.1 wt. % to about 99 wt. % of an additional component, such as about 1 wt. % to about 99 wt. %, about 1 wt. % to about 75 wt. %, about 1 wt. % to about 50 wt. %, about 1 wt. % to about 25 wt. %, about 10 wt. % to about 99 wt. %, about 25 wt. % to about 99 wt. %, about 50 wt. % to about 99 wt. %, about 75 wt. % to about 99 wt. %, about 60 wt. % to about 95 wt. %, or about 75 wt. % to about 95 wt. %.

In some embodiments, the composition comprises a solvent. A concentrate including the sesamol oligomer can be present in a composition with a solvent, or a combination of solvents. A solvent or solvent combination can be chosen so that the sesamol oligomer is soluble in the solvent or solvent combination. Useful solvents include any solvent in which the sesamol oligomer is soluble or can be stably suspended. In some embodiments, a solvent or solvent combination can be selected from an alcohol, a hydrocarbon, a ketone, an ether, an alkylene glycol, a glycol ether, an amide, a nitrile, a sulfoxide, an ester, and any combination thereof.

In some embodiments, a solvent or solvent combination can be selected from methanol, ethanol, propanol, isopropanol, butanol, 2-ethylhexanol, hexanol, octanol, decanol, 2-butoxyethanol, methylene glycol, ethylene glycol, 1, 2-propylene glycol, 1,3-propylene glycol, diethyleneglycol monomethyl ether, diethylene glycol monoethyl ether, ethylene glycol monobutyl ether, ethylene glycol dibutyl ether, pentane, hexane, cyclohexane, kerosene, methylcyclohexane, heptane, decane, dodecane, diesel, toluene, xylene, heavy aromatic naphtha, cyclohexanone, diisobutylketone, diethyl ether, propylene carbonate, N-methylpyrrolidinone, N, N-dimethylformamide, or a combination thereof.

In some embodiments, a solvent or solvent combination can be selected from water soluble or water miscible solvents such glycol-based solvents and hydrophobic or hydrocarbon solvents such as aromatic solvents, paraffinic solvents, or combinations thereof.

Examples of glycol solvents include, but are not limited to, C₁-C₈ glycols such as ethylene glycol, propylene glycol, diethylene glycol, and triethylene glycol, ethers of such glycols, such as diethylene glycol monobutyl ether, diethylene glycol monomethyl ether, diethylene glycol monoethyl ether, diethylene glycol monobutyl ether, triethylene glycol, triethylene glycol monomethyl ether, liquid polyethylene glycol, dipropylene glycol, dipropylene glycol monomethyl ether, dipropylene glycol monoethyl ether, and a low molecular weight polypropylene glycol, and the like, and combinations thereof. Commercial solvents, such as Butyl CARBITOL™ and Butyl CELLOSOLVE™ which include primarily ethylene glycol monobutyl ether.

Examples of hydrophobic or hydrocarbon solvents include, but are not limited to, heavy aromatic naphtha, toluene, ethylbenzene, isomeric hexanes, benzene, xylene, such as ortho-xylene, para-xylene, or meta-xylene, and mixtures of two or more thereof.

If a composition of the present disclosure comprises a solvent, the amount of solvent in the composition is not particularly limited. For example, a composition of the present disclosure may comprise from about 0.1 wt. % to about 99 wt. % of a solvent, such as about 1 wt. % to about 99 wt. %, about 1 wt. % to about 75 wt. %, about 1 wt. % to about 50 wt. %, about 1 wt. % to about 25 wt. %, about 10 wt. % to about 99 wt. %, about 25 wt. % to about 99 wt. %, about 50 wt. % to about 99 wt. %, about 75 wt. % to about 99 wt. %, about 60 wt. % to about 95 wt. %, or about 75 wt. % to about 95 wt. %.

In some embodiments, the composition comprises an additional antioxidant. These optional additional antioxidants can include, for example, a sterically hindered phenol (e.g., 2,6-di-tert-butylphenol), and/or an alkylated diphenylamine. In a concentrated composition (e.g., a concentrate), the one or more optional additional antioxidants can also be at a high concentration along with the sesamol oligomer antioxidant, so that when the concentrate is diluted these components are provided at working concentrations (e.g., ppm).

The compositions disclosed herein may include or exclude an additional antioxidant. For example, the compositions may include or exclude sesamol, a sterically hindered phenol (e.g., 2,6-di-tert-butylphenol), and/or an alkylated diphenylamine. In some embodiments, the composition excludes sesamol.

If a composition of the present disclosure comprises an additional antioxidant, the amount of additional antioxidant in the composition is not particularly limited. For example, a composition of the present disclosure may comprise from about 0.1 wt. % to about 99 wt. % of an additional antioxidant, such as about 1 wt. % to about 99 wt. %, about 1 wt. % to about 75 wt. %, about 1 wt. % to about 50 wt. %, about 1 wt. % to about 25 wt. %, about 10 wt. % to about 99 wt. %, about 25 wt. % to about 99 wt. %, about 50 wt. % to about 99 wt. %, about 75 wt. % to about 99 wt. %, about 60 wt. % to about 95 wt. %, or about 75 wt. % to about 95 wt. %.

A composition of the present disclosure can optionally include one or more additional components, such as additives, that could be beneficial for use in the oxidizable fluids as described herein at a working concentration. These optional additives can include, for example, a chelating agent, a dispersant or detergents, a de-emulsifier, a defoamer, or combinations thereof. In a concentrated composition (e.g., a concentrate), the one or more optional additives can also be at a high concentration along with the sesamol oligomer antioxidant, so that when the concentrate is diluted these components are provided at working concentrations (e.g., ppm).

If a composition of the present disclosure comprises an additive, the amount of additive in the composition is not particularly limited. For example, a composition of the present disclosure may comprise from about 0.1 wt. % to about 50 wt. % of an additive, such as about 1 wt. % to about 50 wt. %, about 10 wt. % to about 50 wt. %, about 20 wt. % to about 50 wt. %, about 30 wt. % to about 50 wt. %, about 40 wt. % to about 50 wt. %, about 0.1 wt. % to about 40 wt. %, about 0.1 wt. % to about 30 wt. %, about 0.1 wt. % to about 20 wt. %, or about 0.1 wt. % to about 10 wt. %.

In some embodiments, the composition comprises from about 0.001 wt. % to about 100 wt. % of the sesamol oligomer, such as from about 0.001 wt. % to about 75 wt. %, about 0.001 wt. % to about 50 wt. %, about 0.001 wt. % to about 25 wt. %, about 0.001 wt. % to about 10 wt. %, about 0.001 wt. % to about 5 wt. %, about 1 wt. % to about 100 wt. %, about 5 wt. % to about 100 wt. %, about 10 wt. % to about 100 wt. %, about 25 wt. % to about 100 wt. %, about 50 wt. % to about 100 wt. %, or about 75 wt. % to about 100 wt. % of the sesamol oligomer.

In some embodiments, the effective amount of the composition is added to the oxidizable fluid to provide a concentration of about 0.0001 wt. % to about 50 wt. % of the sesamol oligomer in the oxidizable fluid. For example, the effective amount of the composition is added to the oxidizable fluid to provide a concentration of about 0.0001 wt. % to about 25 wt. %, about 0.0001 wt. % to about 10 wt. %, about 0.0001 wt. % to about 5 wt. %, about 0.0001 wt. % to about 2.5 wt. %, about 0.0001 wt. % to about 1 wt. %, about 0.0001 wt. % to about 0.1 wt. %, about 0.001 wt. % to about 25 wt. %, about 0.001 wt. % to about 10 wt. %, about 0.001 wt. % to about 5 wt. %, about 0.001 wt. % to about 2.5 wt. %, about 0.001 wt. % to about 1 wt. %, about 0.001 wt. % to about 0.1 wt. %, about 0.01 wt. % to about 25 wt. %, about 0.01 wt. % to about 10 wt. %, about 0.01 wt. % to about 5 wt. %, about 0.01 wt. % to about 2.5 wt. %, about 0.01 wt. % to about 1 wt. %, or about 0.01 wt. % to about 0.1 wt. % of the sesamol oligomer in the oxidizable fluid.

In some embodiments, the effective amount of the composition is added to the oxidizable fluid to provide a concentration of about 1 ppm to about 10,000 ppm of the sesamol oligomer in the oxidizable fluid. For example, the effective amount of the composition is added to the oxidizable fluid to provide a concentration of about 1 ppm to about 5,000 ppm, about 1 ppm to about 3,000 ppm, about 1 ppm to about 2,000 ppm, about 1 ppm to about 1,500 ppm, about 1 ppm to about 1,000 ppm, about 1 ppm to about 750 ppm, about 1 ppm to about 500 ppm, about 1 ppm to about 250 ppm, about 10 ppm to about 10,000 ppm, about 10 ppm to about 5,000 ppm, about 10 ppm to about 3,000 ppm, about 10 ppm to about 2,000 ppm, about 1 ppm to about 1,500 ppm, about 10 ppm to about 1,000 ppm, about 10 ppm to about 750 ppm, about 10 ppm to about 500 ppm, about 10 ppm to about 250 ppm, about 100 ppm to about 10,000 ppm, about 100 ppm to about 5,000 ppm, about 100 ppm to about 3,000 ppm, about 100 ppm to about 2,000 ppm, about 100 ppm to about 1,500 ppm, about 100 ppm to about 1,000 ppm, about 100 ppm to about 750 ppm, about 100 ppm to about 500 ppm, about 250 ppm to about 10,000 ppm, about 250 ppm to about 5,000 ppm, about 250 ppm to about 3,000 ppm, about 250 ppm to about 2,000 ppm, about 250 ppm to about 1,500 ppm, about 250 ppm to about 1,000 ppm, or about 250 ppm to about 750 ppm of the sesamol oligomer in the oxidizable fluid.

While carrying out the methods of the present disclosure, the compositions and/or additives disclosed herein may be applied to an oxidizable fluid batch-wise, continuously, or semi-continuously.

The compositions and/or additives disclosed herein may be added to a fluid using a variety of different application methods known in the art. In some embodiments, the compositions and/or additives may be added continuously or intermittently to the oxidizable fluid, either automatically or manually, by using, for example, chemical injection pumps. The addition may involve dripping, pouring, spraying, pumping, injecting, or otherwise adding the composition and/or additive to the oxidizable fluid.

In some embodiments, the oxidizable fluid is an industrial fluid. In some embodiments, the oxidizable fluid comprises an oil. In some embodiments, the oxidizable fluid is selected from the group consisting of a vegetable oil, a seed oil, a pyrolysis oil, a biodiesel, a hydrocarbon, crude oil, a metal-working fluid, a lubricant, and any combination thereof. In some embodiments, the oxidizable fluid is selected from the group consisting of a vegetable oil, a seed oil, an animal oil, a pyrolysis oil, a biodiesel, a hydraulic fluid, a hydrocarbon, crude oil, a metal-working fluid, a lubricant, and any combination thereof.

The sesamol oligomer antioxidant can be directly added to any type of fluid, either refined or unrefined, to provide antioxidant activity and hinder oxidation of fluid components therein.

In some embodiments, the oxidizable fluid is a pyrolysis oil (i.e., derived from plastic), a crude oil (i.e., derived from petroleum), a hydrocarbon, or combinations thereof. In some embodiments, the oxidizable fluid comprises paraffin compounds, isoparaffins, olefins, diolefins, naphthenes, aromatics, and combinations thereof. In some embodiments, the oxidizable fluid comprises varying amounts of olefins and diolefins.

In some embodiments, the oxidizable fluid is a bio-based fuel or biolubricant. In some embodiments, the oxidizable fluid is a biodiesel, which can be a refined or distilled biodiesel, a crude biodiesel, or a biodiesel that has both crude and refined components. A biodiesel is typically prepared by the esterification of animal and/or vegetable oils. For example, some vegetable oils that are used for preparation of biodiesel include palm oil, soybean oil, rapeseed oil, sesame oil, sunflower oil, grape seed oil, olive oil, and cottonseed oil. Some animal oils that are used for preparation of biodiesel include fish oils and fat from animals, such as pigs and chickens.

In some embodiments, the oxidizable fluid is a vegetable oil, a seed oil, an animal oil, or combinations thereof. Vegetable oils and seed oils are commonly extracted from seeds or other parts of vegetables and fruits. For example, common vegetable and seed oils include palm oil, soybean oil, rapeseed oil, sesame oil, sunflower oil, grape seed oil, olive oil, and cottonseed oil. Common animal oils include fish oil and fat from animals, such as pigs and chickens.

A fluid treated with a composition of the present disclosure can be at any selected temperature, such as ambient temperature or an elevated temperature. For example, the medium may be at a temperature of from about 40 °C to about 250 °C. In some embodiments, the medium may be at a temperature of from about −50 °C to about 300 °C, about 0 °C to about 200 °C, about 10 °C to about 100 °C, or about 20 °C to about 90 °C.

A fluid treated with a composition of the present disclosure has a significantly improved oxidative stability as compared to a fluid that does not comprise a composition of the present disclosure. In some embodiments, the treated fluid undergoes an improvement in oxidative stability of about 10% to about 1000% compared to the untreated fluid, or about 10% to about 100%, about 50% to about 100%, about 50% to about 200%, about 100% to about 200%, about 100% to about 1000%, about 100% to about 800%, about 200% to about 1000%, about 200% to about 800%, about 500% to about 1000%, or about 500% to about 800% compared to the corresponding untreated fluid.

The percent improvement in oxidative stability may be measured as a relative improvement in oxidation stability index (OSI), oxidation induction time (OIT), or oxidation temperature (OT) within the treated fluid compared to the untreated fluid. OSI may be measured by the Rancimat (i.e., a modified EN 14112) test method described herein. OIT may be measured by a PDSC OIT test method described herein. OT may be measured by a PDSC OT test method described herein. A higher OSI, OIT, and/or OT may indicate a higher oxidative stability.

In some embodiments, the composition provides an oxidation stability index (OSI) (at a temperature between 100 °C and 200 °C) of greater than about 50 min, greater than about 75 min, greater than about 100 min, or greater than about 150 min. For example, the composition may provide an OSI of about 50 min to about 500 min, about 50 min to about 250 min, about 75 min to about 500 min, about 75 min to about 250 min, about 100 min to about 500 min, or about 100 min to about 250 min.

In some embodiments, the composition provides an oxidation induction time (OIT) (at a temperature between 100 °C and 200 °C) of greater than about 5 min, greater than about 6 min, greater than about 8 min, greater than about 10 min, greater than about 12 min, greater than about 15 min, greater than about 20 min, or greater than about 25 min. For example, the composition may provide an OIT of about 5 min to about 100 min, about 5 min to about 50 min, about 5 min to about 25 min, about 10 min to about 100 min, about 10 min to about 50 min, or about 10 min to about 25 min.

In some embodiments, the composition provides an oxidation temperature (OT) (at a ramp rate of 10 °C/min) of greater than about 100 °C, greater than about 120 °C, greater than about 140 °C, greater than about 150 °C, greater than about 160 °C, greater than about 180 °C, or greater than about 200 °C. For example, the composition may provide an OT of about 100 °C to about 250 °C, about 100 °C to about 225 °C, about 100 °C to about 200 °C, about 150 °C to about 250 °C, about 150 °C to about 225 °C, or about 150 °C to about 200 °C.

The foregoing may be better understood by reference to the following examples, which are intended for illustrative purposes and are not intended to limit the scope of the disclosure or its application in any way.

### EXAMPLES

### Materials and Methods

Synthesis Method: The following is a representative synthetic scheme that was carried out to produce an embodiment of a sesamol oligomer:

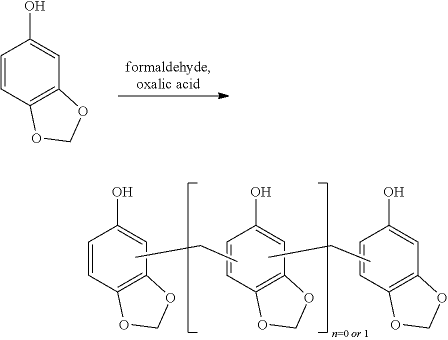

A representative sesamol oligomer was synthesized following a method reported in U.S. Pat. No. 5,275,911. Sesamol was reacted with formaldehyde (formalin solution) in the presence of catalytic amount of oxalic acid to obtain the sesamol oligomer used in the following examples.

Rancimat Method: OSI was measured by Rancimat test using modified Standard Test Method EN 14112, using an air stream that was passed through 7.5 g of a sample of the composition, at a temperature of 100 °C, and at an airflow of 10 L/h, and an antioxidant concentration of 400 ppm.

PDSC OIT method: In a PDSC oxidation induction time method, a sample of pyrolysis oil is heated to a temperature (e.g., about 130 °C, 160 °C, or 170 °C) under increased oxygen pressure (e.g., about 500 psi). Once the target temperature is achieved, the sample remains subjected to the thermo-oxidative conditions in isothermal mode until the oxidation begins. The onset of exothermic oxidation is called the oxidation induction time (OIT). A higher OIT indicates a higher oxidative stability.

PDSC OT method: In a PDSC oxidation temperature method, a sample of pyrolysis oil is heated to 250 °C at a ramp rate of 10 °C/min under increased oxygen pressure (e.g., about 500 psi). The temperature onset of exothermic oxidation is called the oxidation temperature (OT). A higher OT indicates a higher oxidative stability.

The test parameters were as follows: Instrument: DSC25P; Isothermal Temp: about 130 °C, 160 °C, or 170 °C; Purge gas: Oxygen (about 500 PSI); Pan type: Aluminum pan; Sample Mass: about 5 mg; and Ramp about 10 °C/min to 130 °C, 160 °C, or 170 °C.

### Example 1: Performance Evaluation in a Biodegradable Lubricant Based on Unsaturated Trimethylolpropane Esters (TMP Esters) Under Thermo-Oxidative Conditions of PDSC

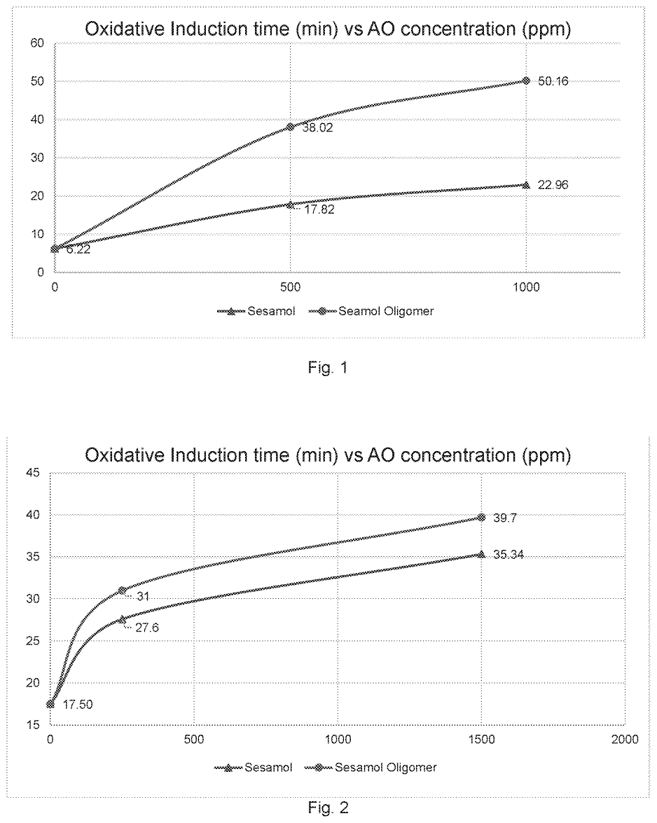

For the evaluation, a vegetable oils-based synthetic ester lubricant oil, Radialube 7364 (Supplier: Oleon, IUPAC name: fatty acids, C18-unsatd., diesters and triesters with trimethylolpropane, CAS No.: 1335202-94-2), was utilized. The PDSC results are presented in Table 1 and FIG. 1 below. The untreated TMP ester-based lubricant oil exhibited an oxidative stability (PDSC, OIT measured at 130 °C) of 6.22 minutes. When sesamol was used at 500 ppm, the induction time increased to 17.82 minutes. A more substantial increase in induction time, reaching 38.02 minutes, was observed when sesamol oligomer was used at 500 ppm. Depending on the treatment level (500-1000 ppm), the sesamol oligomer extended the induction time by 511-706% relative to the induction time without an antioxidant. The data indicate that the addition of sesamol oligomer significantly enhances the oxidative stability of unsaturated TMP ester lubricant compared to sesamol at the same treatment level.

TABLE 1
Isothermal PDSC OIT results for Radialube 7364 with 500
ppm and 1000 ppm of sesamol and sesamol oligomer

| | | AO conc. | Temp | OIT | % |
|---|---|---|---|---|---|
| Oil | Antioxidant | (ppm) | (°C) | (min) | improvement |
| Radialube | Blank | 0 | 130 | 6.22 | 0 |
| 7364 | Sesamol | 500 | 130 | 17.82 | 187 |
| | Sesamol | 500 | 130 | 38.02 | 511 |
| | oligomer | | | | |
| | Sesamol | 1000 | 130 | 22.96 | 269 |
| | Sesamol | 1000 | 130 | 50.16 | 706 |
| | oligomer | | | | |

### Example 2: Performance Evaluation in a Biodegradable Lubricant Based on Saturated Trimethylolpropane Esters (TMP Esters) Under Thermo-Oxidative Conditions of PDSC

Vegetable oil-based synthetic ester lubricant oil, Radialube 7257 (Supplier: Oleon, IUPAC name: fatty acids, complex ester with 2-ethyl-2-(hydroxymethyl)-1,3-propanediol) was used for evaluation. PDSC results are shown in Table 2 below. The untreated Radialube 7257 oil had an oxidative stability (PDSC, OIT measured at 170 °C) of 12 minutes. When sesamol was added at 500 ppm, the induction time increased to 16 minutes. A further increase in the induction time to 19 minutes was observed when sesamol oligomer was used at 500 ppm. Sesamol oligomer extended the induction time by 61% compared to the induction time without an antioxidant.

TABLE 2
The OIT results of isothermal PDSC for Radialube 7257
with 500 ppm and 1000 ppm of sesamol and sesamol oligomer.

| | | AO conc. | Temp | OIT | % |
|---|---|---|---|---|---|
| Oil | Antioxidant | (ppm) | (°C) | (min) | improvement |
| Radialube | Blank | 0 | 170 | 12 | 0 |
| 7257 | Sesamol | 500 | 170 | 16 | 38 |
| | Sesamol | 500 | 170 | 19 | 61 |
| | oligomer | | | | |

### Example 3: Performance Evaluation in a Plastic Pyrolysis Oil-1 Under Thermo-Oxidative Conditions of PDSC

Plastic pyrolysis oil-1 was utilized for evaluation. The PDSC results are presented in Table 3 and FIG. 2. The untreated plastic pyrolysis oil-1 exhibited an oxidative stability (PDSC, OIT measured at 170 °C) of 17.5 minutes. When sesamol was added at a concentration of 250 ppm, the induction time increased to 27.6 minutes. A more significant increase in induction time to 31 minutes was observed when sesamol oligomer was used at the same concentration of 250 ppm. Depending on the treatment level (250-1500 ppm), the sesamol oligomer extended the induction time by 77-126% relative to the induction time without an antioxidant. Sesamol oligomer demonstrated a notable enhancement in the oxidative stability of plastic pyrolysis oil-1 compared to sesamol at the same treatment level.

TABLE 3
Isothermal PDSC OIT results for pyrolysis oil-1 with 250
and 1500 ppm of sesamol and sesamol oligomer

| | | AO conc. | Temp | OIT | % |
|---|---|---|---|---|---|
| Oil | Antioxidant | (ppm) | (°C) | (min) | improvement |
| Plastic | Blank | 0 | 170 | 17.50 | 0 |
| pyrolysis | Sesamol | 250 | 170 | 27.6 | 57 |
| oil-1 | Sesamol | 250 | 170 | 31 | 77 |
| | oligomer | | | | |
| | Sesamol | 1500 | 170 | 35.34 | 102 |
| | Sesamol | 1500 | 170 | 39.7 | 126 |
| | oligomer | | | | |

### Example 4: Performance Evaluation in a Plastic Pyrolysis Oil-2 Under Thermo-Oxidative Conditions of Rancimat Test

Plastic pyrolysis oil-2 was utilized for evaluation. The Rancimat results are presented in Table 4 below. Untreated plastic pyrolysis oil-2 had an oxidative stability index (Rancimat, OSI measured at 120 °C) of 53.4 minutes. When 2,6-DTBP and sesamol were added at 250 ppm, the induction time increased to 94.2 and 105.6 minutes, respectively. A further increase in induction time to 117 minutes was observed when sesamol oligomer was used at 250 ppm. The sesamol oligomer extended the induction time by 119% compared to the induction time without an antioxidant.

TABLE 4
OSI results for pyrolysis oil-2 with 250 ppm of 2,6-DTBP
sesamol, and sesamol oligomer

| | | AO conc. | Temp | OSI | % |
|---|---|---|---|---|---|
| Oil | Antioxidant | (ppm) | (°C) | (min) | improvement |
| Plastic | Blank | 0 | 120 | 53.4 | 0 |
| pyrolysis | Sesamol | 250 | 120 | 105.6 | 98 |
| oil-2 | Sesamol | 250 | 120 | 117 | 119 |
| | oligomer | | | | |
| | 2,6-DTBP | 250 | 120 | 94.2 | 76 |

### Example 5: Performance Evaluation in a Plastic Pyrolysis Oil-1 Under Thermo-Oxidative Conditions of Rancimat

Plastic pyrolysis oil-1 was used for evaluation. The Rancimat results are detailed in Table 5 below. The untreated plastic pyrolysis oil-1 exhibited an oxidative stability index (OSI measured at 160 °C) of 166.2 minutes. When treated with 250 ppm of 2,6-DTBP and sesamol, the induction times increased to 393.6 and 361.2 minutes, respectively. Notably, the use of sesamol oligomer at 250 ppm resulted in a more significant increase in the induction time to 430.8 minutes. The sesamol oligomer extended the induction time by 159% compared to the baseline induction time without an antioxidant.

TABLE 5
OSI results for pyrolysis oil-1 with 250 ppm of 2,6-DTBP,
sesamol, and sesamol oligomer

| | | AO conc. | Temp | OSI | % |
|---|---|---|---|---|---|
| Oil | Antioxidant | (ppm) | (°C) | (min) | improvement |
| Plastic | Blank | 0 | 160 | 166.2 | 0 |
| pyrolysis | Sesamol | 250 | 160 | 393.6 | 137 |
| oil-1 | Sesamol | 250 | 160 | 430.8 | 159 |
| | oligomer | | | | |
| | 2,6-DTBP | 250 | 160 | 361.2 | 117 |

### Example 6: Performance Evaluation in a Biodegradable Lubricant Based on Unsaturated Trimethylolpropane Esters (TMP Esters) Under Thermo-Oxidative Conditions of PDSC

Radialube 7364, a vegetable oils-based synthetic ester lubricant (Supplier: Oleon, IUPAC name: fatty acids, C18-unsatd., diesters and triesters with trimethylolpropane, CAS No.: 1335202-94-2), was used for testing. PDSC results are shown in Table 6. Untreated Radialube 7364 had an oxidation temperature of 120 °C (PDSC, OT at 10 °C/min). Adding sesamol at 1000 ppm increased the oxidation temperature to 162 °C. Using sesamol oligomer at the same concentration raised it further to 192 °C, a 61% increase from the untreated oil.

TABLE 6
Oxidation Temperature (°C) Results for Radialube 7364
Lubricant with 1000 ppm of Sesamol and Sesamol Oligomer

| | | AO | Ramp | | % |
|---|---|---|---|---|---|
| | | conc. | Rate | OT | improve- |
| Oil | Antioxidant | (ppm) | (°C) | (temp) | ment |
| Radialube | Blank | 0 | 10 C/min | 120 C | 0 |
| 7364 | Sesamol | 1000 | 10 C/min | 162 C | 35 |
| | Sesamol | 1000 | 10 C/min | 192 C | 61 |
| | oligomer | | | | |

### Example 7: Performance Evaluation in a Biodegradable Lubricant Based on Unsaturated Trimethylolpropane Esters (TMP Esters) Under Thermo-Oxidative Conditions PDSC

Radialube 7361, a vegetable oil-based synthetic ester lubricant from Oleon (CAS No.: 57675-44-2), was evaluated. Table 7 shows the PDSC results. The untreated oil had an oxidation temperature of 158 °C at a 10 °C/min ramp rate. Adding 1000 ppm sesamol raised it to 174 °C, while 1000 ppm sesamol oligomer increased it further to 191 °C, a 21% increase compared to the untreated oil.

TABLE 7
Oxidation temperature (°C) data for Radialube 7361
lubricant containing 1000ppm of sesamol and sesamol oligomer

| | | AO | Ramp | | % |
|---|---|---|---|---|---|
| | | conc. | Rate | OT | improve- |
| Oil | Antioxidant | (ppm) | (°C) | (temp) | ment |
| Radialube | Blank | 0 | 10 C/min | 158 | 0 |
| 7361 | Sesamol | 1000 | 10 C/min | 174 | 10 |
| | Sesamol | 1000 | 10 C/min | 191 | 21 |
| | oligomer | | | | |

### Example 8: Performance Evaluation in Biodiesel Under Thermo-Oxidative Conditions of PDSC

Canola oil-based biodiesel was evaluated, with PDSC results shown in Table 8. Untreated biodiesel had an oxidative stability of 7.96 min at 140 °C. Sesamol at 1000 ppm increased the induction time to 11.48 min, while sesamol oligomer at 1000 ppm raised it to 13.86 min, a 74% increase compared to untreated biodiesel.

TABLE 8
OIT results of isothermal PDSC for biodiesel with 1000
ppm sesamol and sesamol oligomer

| | | AO conc. | Temp | OIT | % |
|---|---|---|---|---|---|
| Oil | Antioxidant | (ppm) | (°C) | (min) | improvement |
| Biodiesel | Blank | 0 | 140 | 7.96 | 0 |
| | Sesamol | 1000 | 140 | 11.48 | 44 |
| | Sesamol | 1000 | 140 | 13.86 | 74 |
| | oligomer | | | | |

All of the compositions and methods disclosed and claimed herein can be made and executed without undue experimentation in light of the present disclosure. While this invention may be embodied in many different forms, there are described in detail herein specific preferred embodiments of the invention. The present disclosure is an exemplification of the principles of the invention and is not intended to limit the invention to the particular embodiments illustrated. In addition, unless expressly stated to the contrary, use of the term “a” is intended to include “at least one” or “one or more.” For example, “a sesamol oligomer” is intended to include “at least one sesamol oligomer” or “one or more sesamol oligomers.”

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges (including all fractional and whole values) subsumed therein.

Any composition disclosed herein may comprise, consist of, or consist essentially of any element, reaction product, component, compound and/or additive disclosed herein or any combination of two or more of the elements, reaction products, components, compounds or additives disclosed herein.

Any method disclosed herein may comprise, consist of, or consist essentially of any method step disclosed herein or any combination of two or more of the method steps disclosed herein.

The transitional phrase “comprising,” which is synonymous with “including,” “containing,” or “characterized by,” is inclusive or open-ended and does not exclude additional, un-recited elements, components, ingredients and/or method steps.

The transitional phrase “consisting of” excludes any element, component, ingredient, and/or method step not specified in the claim.

The transitional phrase “consisting essentially of” limits the scope of a claim to the specified elements, components, ingredients and/or steps, as well as those that do not materially affect the basic and novel characteristic(s) of the claimed invention.

Unless specified otherwise, all molecular weights referred to herein are weight average molecular weights and all viscosities were measured at 25 °C with neat (not diluted) polymers.

As used herein, the term “about” refers to the cited value being within the errors arising from the standard deviation found in their respective testing measurements, and if those errors cannot be determined, then “about” may refer to, for example, within 5% of the cited value.

Furthermore, the invention encompasses any and all possible combinations of some or all of the various embodiments described herein. It should also be understood that various changes and modifications to the presently preferred embodiments described herein will be apparent to those skilled in the art. Such changes and modifications can be made without departing from the spirit and scope of the invention and without diminishing its intended advantages. It is therefore intended that such changes and modifications be covered by the appended claims.

What is claimed is:

1. A method for stabilizing an oxidizable fluid, comprising:
adding a composition comprising a sesamol oligomer to the oxidizable fluid.

2. The method of claim 1, wherein the sesamol oligomer is of formula I: 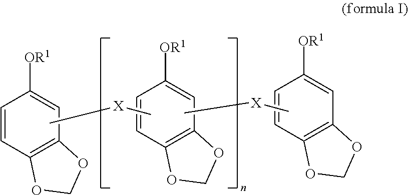
or a salt thereof, wherein:
each R¹ is independently hydrogen or C₁-C₆ alkyl,
each X is independently a bond or C₁-C₆ alkylene, and
n is 0 or an integer of 1 to 20.

3. The method of claim 1, wherein the sesamol oligomer is selected from the group consisting of: 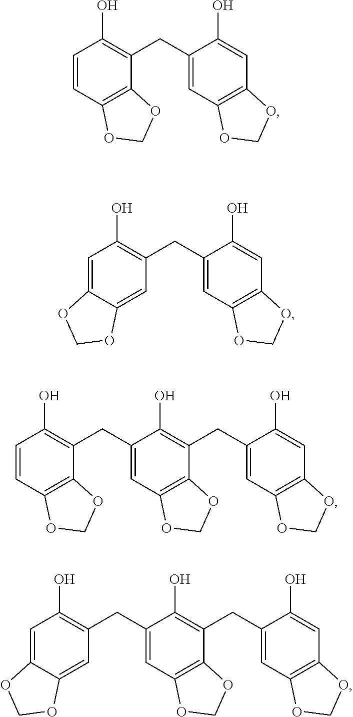 [SMILES: Oc1cc2c(cc1Cc1c(O)ccc3c1OCO3)OCO2 ; Oc1cc2c(cc1Cc1cc3c(cc1O)OCO3)OCO2 ; Oc1cc2c(cc1Cc1c(O)c(Cc3c(O)ccc4c3OCO4)cc3c1OCO3)OCO2 ; Oc1cc2c(cc1Cc1cc3c(c(Cc4cc5c(cc4O)OCO5)c1O)OCO3)OCO2]
and any combination thereof.

4. The method of claim 1, further comprising heating the oxidizable fluid to a temperature of about 20 °C to about 250 °C and/or further comprising adding from about 1 ppm to about 10,000 ppm of the sesamol oligomer to the oxidizable fluid.

5. The method of claim 1, further comprising producing the sesamol oligomer by reacting sesamol with an aldehyde in the presence of a catalyst.

6. The method of claim 5, wherein the aldehyde comprises formaldehyde, acetaldehyde, benzaldehyde, vanillin, salicylaldehyde, furfural, glyoxal, glyoxylic acid, or any combination thereof, and/or wherein the catalyst comprises oxalic acid, hydrochloric acid, orthophosphoric acid, sulfuric acid, sulfonic acid, and any combination thereof.

7. The method of claim 1, wherein the composition comprises from about 1 wt. % to about 100 wt. % of the sesamol oligomer.

8. The method of claim 1, wherein the composition further comprises an additional component selected from the group consisting of a solvent, an additional antioxidant, and any combination thereof.

9. The method of claim 8, wherein the solvent is selected from the group consisting of an alcohol, a hydrocarbon, a ketone, an ether, an alkylene glycol, a glycol ether, an amide, a nitrile, a sulfoxide, an ester, and any combination thereof.

10. The method of claim 8, wherein the composition comprises from about 1 wt. % to about 99 wt. of the additional component.

11. The method of claim 1, wherein the composition excludes sesamol and/or wherein the method excludes adding sesamol to the oxidizable fluid.

12. The method of claim 1, wherein the oxidizable fluid is selected from the group consisting of a vegetable oil, a seed oil, a pyrolysis oil, a biodiesel, a hydrocarbon, crude oil, a metal-working fluid, a lubricant, and any combination thereof.

13. The method of claim 1, further comprising preventing or inhibiting oxidation of the oxidizable fluid.

14. A composition, comprising:
an oxidizable substrate and a sesamol oligomer,
wherein the oxidizable substrate is an oxidizable fluid selected from the group consisting of a vegetable oil, a seed oil, a pyrolysis oil, a biodiesel, a hydrocarbon, crude oil, a metal-working fluid, a lubricant, and any combination thereof.

15. The composition of claim 14, wherein the sesamol oligomer is of formula I: 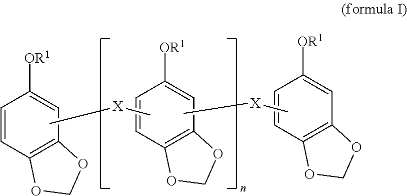
or a salt thereof, wherein:
each R¹ is independently hydrogen or C₁-C₆ alkyl,
each X is independently a bond or C₁-C₆ alkylene, and
n is 0 or an integer of 1 to 20.

16. The composition of claim 14, wherein the sesamol oligomer is selected from the group consisting of: 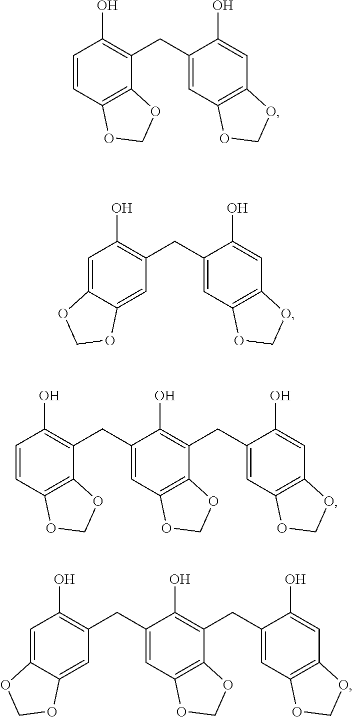 [SMILES: Oc1cc2c(cc1Cc1c(O)ccc3c1OCO3)OCO2 ; Oc1cc2c(cc1Cc1cc3c(cc1O)OCO3)OCO2 ; Oc1cc2c(cc1Cc1c(O)c(Cc3c(O)ccc4c3OCO4)cc3c1OCO3)OCO2 ; Oc1cc2c(cc1Cc1cc3c(c(Cc4cc5c(cc4O)OCO5)c1O)OCO3)OCO2]
and any combination thereof.

17. The composition of claim 14, wherein the sesamol oligomer is produced by reacting sesamol with an aldehyde in the presence of a catalyst.

18. The composition of claim 14, wherein the composition comprises from about 0.1 wt. % to about 99 wt. % of the sesamol oligomer.

19. The composition of claim 14, wherein the composition further comprises an additional component selected from the group consisting of a solvent, an additional antioxidant, and combinations thereof.

20. The composition of claim 14, wherein the composition excludes sesamol.
